(19) United States
(12) Patent Application Publication
(10) Pub. No.: US 2007/0255335 A1
(43) Pub. Date: Nov. 1, 2007

(54) Controller for gastric constriction device with selectable electrode configurations

(75) Inventors: Timothy P. Herbert, Maple Grove, MN (US); Warren L. Starkebaum, Plymouth, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/414,502

(22) Filed: Apr. 28, 2006

(51) Int. Cl. A61N 1/00

(52) U.S. Cl. 607/40

(57) ABSTRACT

This disclosure describes an implantable medical device that delivers electrical stimulation to a patient in combination with restriction ingestion of food by the patient to treaty obesity. The implantable medical device includes a gastric constriction device, such as a hydraulic or mechanical gastric band having a plurality of electrodes integrally formed thereon. A controller includes a control unit to control the degree of gastric constriction of the gastric constriction device and a pulse generator to control delivery of electrical stimulation to the patient. The control unit and pulse generator are enclosed within a housing implanted within the patient. By enclosing the control unit and pulse generator within a common housing, the trauma experienced by the patient during the implantation procedure may be substantially reduced. Additionally, the cost of the controller may be less than the cost of purchasing a control unit and a pulse generator separately.

### TECHNICAL FIELD

The invention relates to medical devices and, more particularly, to devices for the treatment of obesity.

### BACKGROUND

Various surgical techniques have been developed to treat morbid obesity. One of these techniques involves use of a gastric banding device. Gastric bands are typically constructed in the form of a hollow tube that can be inserted through a laparoscopic cannula to completely encircle an upper end of the stomach. The band is constricted to limit the passage of food into the lower stomach.

There are two basic types of gastric bands: hydraulic bands and mechanical bands. With a mechanical gastric band, the degree of gastric constriction is adjusted mechanically by a motor that tightens or loosens the band about the stomach. A hydraulic band is typically fabricated from an elastomer, such as silicone rubber. The degree of gastric constriction depends upon the amount of fluid injected into the hydraulic band. For a hydraulic band, a fluid reservoir contains an amount of fluid. A hypodermic needle may be used to percutaneously inject and withdraw fluid to and from the reservoir.

Alternatively, a pump unit may be implanted within the patient. The pump unit pumps fluid from the reservoir to the band to reduce the size of the stoma opening and pumps fluid from the gastric band back to the reservoir to enlarge the size of the stoma opening. For a hydraulic band, a control unit implanted within the patient controls the pump and thus the size of the stoma opening. For a mechanical pump, an implanted control unit controls the motor to tighten and loosen the mechanical band.

Electrical stimulation of the gastrointestinal tract also has been used to treat obesity. Typically, electrical stimulation involves the use of electrodes implanted in the wall of a target organ, such as the stomach. The electrodes are electrically coupled to an implanted or external pulse generator via implanted or percutaneous leads. The pulse generator delivers a stimulation waveform via the leads and electrodes. For example, electrical stimulation of the stomach may be effective in reducing the desire of the patient to eat by inducing a feeling of fullness or nausea. Alternatively, electrical stimulation of the small intestine may be effective in reducing food absorption by moving the food through the small intestine more quickly, i.e., increasing gastric motility.

### SUMMARY

In general, the invention is directed to an implantable medical device that delivers electrical stimulation to a patient in combination with restricting ingestion of food by the patient to treat obesity. The implantable medical device includes a gastric constriction device, such as a hydraulic or mechanical gastric band, and an array of electrodes integrally formed in the gastric constriction device. An implantable motor or pump may be provided to adjust the gastric constriction device to restrict food intake. An implantable pulse generator delivers stimulation energy via one or more of the electrodes integrated in the constriction device to induce a sensation of fullness or nausea. The implantable motor or pump and the pulse generator are enclosed within a common housing implanted within the patient. The motor or pump, the pulse generator, and the housing containing the motor or pump and the pulse generator are collectively referred to herein as a “controller.“

Enclosing the motor or pump and the pulse generator within a common housing enables a surgeon to implant the common housing within a single subcutaneous pocket created within the patient, thereby reducing trauma experienced by the patient in comparison to systems in which two different housings enclose the motor or pump and the pulse generator, respectively. Additionally, the electrical components of the motor or pump and the pulse generator may be fabricated on a single circuit board to reduce size and cost. Moreover, some of the electrical components may be used for both gastric band control and stimulation control, avoiding duplication of electronics.

The integration of an array of stimulation electrodes within a gastric constriction device permits a clinician to select a combination of gastric constriction and electrical stimulation to treat obesity. The implantable pulse generator may be programmed to drive a selected combination of electrodes from the integrated electrode array, or multiple electrode combinations on a time-interleaved basis. The electrodes are distributed at various positions around the gastric constriction device, permitting the clinician to test stimulation at different stimulation sites and select the most effective electrode combination or combinations. In some embodiments, additional electrodes may be provided outside of the constriction device.

In one embodiment, the invention provides an implantable medical device comprising a gastric constriction device configured to constrict a portion of a gastrointestinal tract of a patient, a plurality of electrodes carried by the gastric constriction device, a housing implanted in the patient, and a controller within the housing that controls a degree of gastric constriction provided by the gastric constriction device, selects one or more of the electrodes, and delivers electrical stimulation energy to the patient via the selected electrodes.

In another embodiment, the invention provides a method comprising constricting a portion of a gastrointestinal tract of a patient using a gastric constriction device, wherein the gastric constriction device carries a plurality of electrodes, delivering electrical stimulation energy to the constricted portion of the gastrointestinal tract via a selected subset of the electrodes, and controlling the gastric constriction device and the delivery of electrical stimulation energy via a common controller.

In an additional embodiment, the invention provides an external control device for controlling an implantable gastric constriction device and an implantable electrical stimulation generator, the external control device comprising a processor that generates control signals to control operation of the implantable gastric constriction device and the implantable electrical stimulation generator, and a wireless telemetry interface that communicates the control signals to at least one control unit that controls the implantable gastric constriction device and the implantable electrical stimulation generator. In another embodiment, the invention provides a method for controlling an implantable gastric constriction device and an implantable electrical stimulation generator, the method comprising generating control signals to control operation of the implantable gastric constriction device and the implantable electrical stimulation generator, and communicating the control signals by wireless telemetry to at least one control unit that controls the implantable gastric constriction device and the implantable electrical stimulation generator.

In various embodiments, the invention may provide one or more advantages. For example, in addition to delivering electrical stimulation to a patient via a subset of electrodes selected from an array of electrodes integrally formed in a gastric constriction device that restricts the food intake of the patient, the invention includes a common housing implanted within the patient that contains a motor or pump for controlling the degree of gastric constriction provided by the gastric constriction device and a pulse generator for controlling delivery of electrical stimulation to the patient via the selected electrodes.

Circuitry associated with the motor or pump and the pulse generator may be fabricated on a single circuit board and, thus, share at least one electrical component, e.g., a processor and/or memory. As a result, the common housing may be substantially smaller and cost less than two different housings that separately contain a motor or pump and a pulse generator, respectively. Furthermore, the common housing may be implanted within the patient using fewer incisions and requiring less space. In this manner, the common housing may reduce the trauma experienced by the patient during the implantation process.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating an example implantable system for delivering electrical stimulation to a patient in combination with gastric banding.

FIG. 2 is a block diagram illustrating an example controller of the system in FIG. 1.

FIG. 3 is a lengthwise cross-sectional side view of the gastric constriction device of FIG. 1.

FIG. 4 is a top view of the gastric constriction device of FIG. 1 in a ring configuration.

FIGS. 5A-5D are plan views of an interior side of the gastric constriction device of FIG. 3, illustrating various example electrode patterns.

FIG. 6 is a block diagram illustrating an example external programmer in wireless communication with the controller of FIG. 1 that allows a patient or clinician to control delivery of electrical stimulation, the degree of gastric constriction, or both.

FIG. 7 is a schematic diagram illustrating an additional example implantable system for delivering electrical stimulation to a patient in combination with gastric banding.

FIG. 8 is a schematic diagram illustrating a further example implantable system for delivering electrical stimulation to a patient in combination with gastric banding.

FIG. 9 is a flow chart illustrating a technique for delivering electrical stimulation to a patient in combination with gastric banding in accordance with an embodiment of the invention.

### DETAILED DESCRIPTION

Obesity is an increasing problem for many people, as individuals are consuming more calories and exercising less frequently than necessary to maintain body weight. In some cases, traditional methods for reducing body weight in obese patients may be ineffective, impractical, or potentially dangerous. In accordance with an embodiment of the invention, an implantable medical device includes a gastric constriction device, such as a hydraulic or mechanical gastric band and an array of electrodes integrally formed in the gastric constriction device. An implantable motor or pump may be provided to adjust the gastric constriction device to restrict food intake. An implantable pulse generator delivers stimulation energy via one or more of the electrodes integrated in the constriction device to reduce appetite and induce a sensation of fullness or nausea. The implantable motor or pump and the pulse generator are enclosed within a common housing implanted within the patient. The motor or pump, the pulse generator, and the housing containing the motor or pump and the pulse generator are collectively referred to herein as the “controller.”

The gastric constriction device restricts the ingestion of food to reduce caloric intake by forming a stoma opening in the stomach by encircling and partitioning the stomach into an upper and a lower stomach. Delivering electrical stimulation to the patient via selected electrodes integrally formed with the gastric constriction device may be effective in reducing the desire of the patient to eat and prolonging a feeling of satiety in the patient in response to food intake. Stimulation may modulate or disrupt the normal myoelectric activity of the stomach or small intestine depending on where the stimulation electrodes are placed and the stimulation parameters utilized. Changes in myoelectric activity may, in turn, result in changes in gastric distention or gastric emptying, or in the case of the small intestine, changes in the rate at which food contents move through the small intestine. These effects, i.e., changes in myoelectric or gastrointestinal (GI) motor activity are interpreted by the brain as feelings of early satiety, reduced appetite, or mildly unpleasant upper GI symptoms such as nausea. Nausea or other mildly unpleasant upper GI symptoms may be intentionally induced to produce aversive consequences to overeating or other dyspeptic symptoms. Changes in myoelectric or gastrointestinal motor activity, singly or in combination, may lead to reduced food intake and increased satiety by the patient, and over time, reduced body weight. Electrical stimulation may alternatively or additionally be formulated to vary gastric motility, i.e., increase or decrease gastric motility. In this manner, a gastric constriction device with integrated electrical stimulation electrodes may more completely treat obesity by limiting food intake and varying gastric motility, providing a multi-pronged therapy for treatment of obesity.

The implantable motor or pump and the pulse generator may be contained in separate housings. For example, the housings may be implanted within two different subcutaneous pockets created in the patient or, alternatively, within the same subcutaneous pocket. In accordance with an embodiment of the invention, however, a controller includes a housing that encloses the implantable motor or pump and the pulse generator. In this manner, a single controller is used to control both a gastric constriction device, such as a band, and delivery of electrical stimulation, e.g., via electrodes carried by the band.

In some embodiments, circuitry associated with the motor or pump and the pulse generator may be fabricated on a single circuit board. In addition, circuitry for controlling the motor or pump and the pulse generator may share at least one electrical component, e.g., a processor and/or memory, thereby reducing redundancy and duplication of electronics. As a result, the common housing may be substantially smaller in size and cost less than a motor or pump and a pulse generator contained within separate housings. Additionally, the common housing may be implanted within the patient using fewer incisions and requiring less space. In this manner, the common housing may reduce the trauma experienced by the patient during the implantation procedure.

The gastric constriction device delivers electrical stimulation to the restricted portion of the gastrointestinal tract via one or more electrodes selected from a plurality of electrodes integrally formed with the gastric constriction device. The electrodes may be molded into the gastric constriction device such that each electrode has at least a partially exposed surface that contacts the patient when the gastric constriction device is implanted. The electrodes may be positioned circumferentially around the restricted portion of the gastrointestinal tract with even or irregular spacing and are coupled to the controller. More specifically, the electrodes are coupled to the pulse generator within the controller via corresponding electrode leads. In addition to the pulse generator, the controller may also include a switch matrix to select one or more of the electrodes to deliver electrical stimulation to the patient.

A clinician may test all or at least a portion of the possible electrode combinations of electrodes within the electrode array embedded in the gastric constriction device in order to identify an efficacious combination of electrodes and associated polarities. An electrode combination refers to a subset of electrodes and the polarities of electrodes in the selected subset. A single electrode combination may include a number of adjacent electrodes that deliver electrical stimulation to a localized region, or electrodes arranged in a staggered configuration that deliver electrical stimulation to a more general region. Each electrode combination must include at least one anode and one cathode. In some embodiments, however, the common housing functions as an electrode, providing a unipolar arrangement.

More than one electrode combination may be selected to deliver electrical stimulation to the patient. In this case, multiple combinations of electrodes may be used on a time-interleaved or sequential basis to deliver stimulation to different stimulation sites. In addition, multiple stimulation programs may be delivered via one or more electrode combinations. A stimulation program generally refers to an electrode combination and a set of stimulation parameters including, for example, current or voltage amplitude, stimulation pulse width, and stimulation pulse rate. As mentioned previously, additional electrodes may be implanted independently of the gastric constriction device elsewhere in the gastrointestinal tract, e.g., in the upper stomach, lower stomach, small intestine, and/or duodenum.

For example, the constriction device may be positioned about the proximal stomach, and a pair of stimulation electrodes may be positioned in the distal stomach (antrum) a few centimeters proximal to the pylorus. In this case, the constriction device serves to limit food intake, and stimulation of the antrum using suitable stimulator parameters can delay or retard gastric emptying and result in a prolonged sensation of fullness, leading to reduced food intake and eventual weight loss. As another example, applying stimulation to the proximal stomach may distend the stomach in the fasted state, thereby causing a feeling of fullness prior to meals. Consequently, applying stimulation at various locations in the gastrointestinal tract may reduce appetite, prolong satiety, or both thereby further enhancing or promoting weight loss, and enhancing the effect of a constriction device.

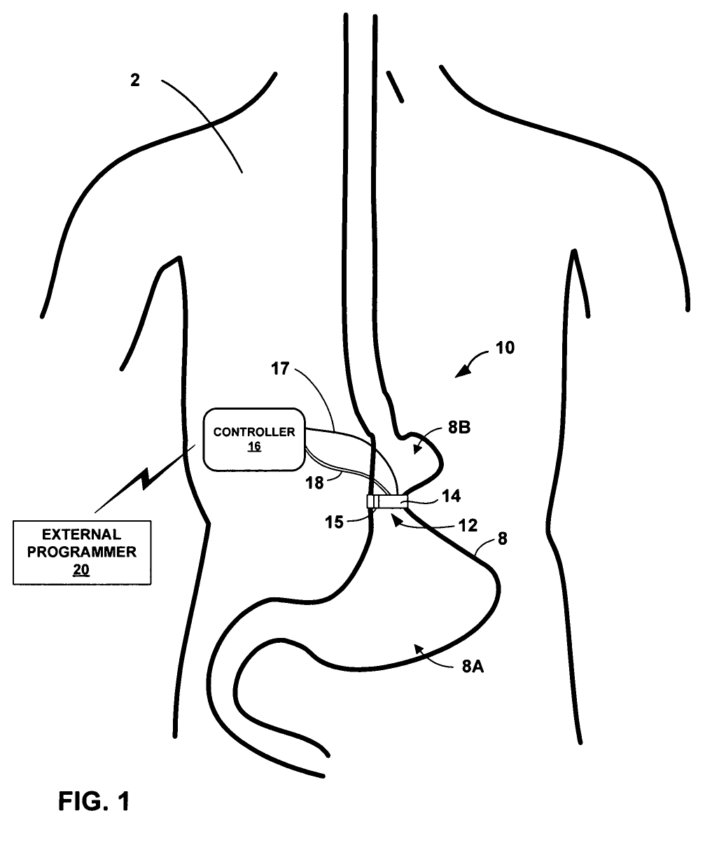

FIG. 1 is a schematic diagram illustrating an implantable medical system 10 configured for the treatment of obesity. In particular, FIG. 1 illustrates system 10 implanted within a torso of a patient 2 in which stomach 8 is visible. System 10 includes a gastric constriction device 12 with electrodes 14 (not shown) integrally formed thereon, a controller 16, and an external programmer 20 in wireless communication with controller 16. Controller 16 controls the degree of gastric constriction of gastric constriction device 12 and delivery of electrical stimulation to patient 2 via electrodes 14. Controller 16 may have a reduced size, relative to two separate controller housings. The reduced size may reduce trauma and discomfort experienced by patient 2 during and after the implantation procedure. In addition, the need to subcutaneously implant only one controller may reduce the time and complexity of the implantation procedure.

In general, system 10 treats obesity by controlling the degree of gastric constriction in combination with delivering electrical stimulation to patient 2 via one or more electrodes selected as a subset of the plurality of electrodes 14. As shown in FIG. 1, gastric constriction device 12 forms a stoma opening in stomach 8 by encircling and partitioning stomach 8 into an upper stomach 8A and a lower stomach 8B. The degree of gastric constriction provided by gastric constriction device 12 (and thus the size of the stoma opening) is designed to limit the ingestion of food and reduce caloric intake so that patient 2 loses weight while permitting the ingestion of water and the minimum amount of caloric energy necessary to prevent malnourishment.

In addition to or, more particularly, in combination with limiting food intake, electrodes 14 (not shown) deliver electrical stimulation to patient 2 to suppress appetite. For example, delivering electrical stimulation to the restricted portion of stomach 8 may induce a feeling of fullness or nausea. In addition, electrical stimulation may be effective by varying gastric motility. For example, electrical stimulation may be formulated to reduce food absorption by moving the food through the GI tract more quickly. In another example, electrical stimulation may be formulated to delay gastric emptying so patient 2 experiences a sensation of fullness more quickly. Consequently, system 10 may provide a multi-pronged approach for treating obesity by limiting food intake and varying increasing gastric motility, i.e., the rate at which food moves through the stomach, small intestine, or elsewhere in the gastroesophageal tract.

Although gastric constriction device 12 is shown in FIG. 1 positioned around the top end (fundus) of stomach 8 in a position commonly associated with an adjustable gastric banding (AGB) procedure, the band may also be placed vertically, as for a vertical banded gastroplasty (VBG), or in any other position designed to reduce food intake. The band may also be used with other portions of the gastrointestinal (GI) tract, such as the esophagus or intestines.

Gastric constriction device 12 may be any type of gastric constriction device, such as a hydraulic gastric band, a mechanical gastric band, or other type of gastric constriction device designed to restrict or limit food intake by constriction of the stomach or, more generally, the gastrointestinal tract. Controller 16 may include any combination of circuitry and/or mechanical hardware designed to adjust the degree of constriction applied by gastric constriction device 12. The combination of circuitry and/or mechanical hardware used to actuate gastric constriction device 12 and deliver electrical stimulation is generally referred to herein as a controller or control unit.

For example, when gastric constriction device 12 comprises a hydraulic gastric band, the degree of gastric constriction depends upon the amount of fluid, such as saline or an expandable fluid, injected into the band. Accordingly, controller 16 may include a fluid reservoir and an injection port for injecting or withdrawing fluid from the reservoir by inserting a needle into the injection port. In this case, adjustment of the band requires puncture of the patient's skin resulting in discomfort for the patient and an increased risk of infection. In order to eliminate additional medical visits and discomfort, controller 16 may include a pump unit and control circuitry to hydraulically tighten and loosen the band.

When controller 16 includes a pump unit, the pump unit pumps fluid from the reservoir through a conduit 18 to the band to reduce the size of the stoma opening. The pump unit may also pump fluid from the gastric band back to the reservoir to enlarge the size of the stoma opening. Thus, the degree of gastric occlusion provided by the band can be adjusted by varying the amount of fluid in the band without requiring a medical visit. In some embodiments, controller 16 may dynamically adjust the degree of gastric constriction based on a sensed physiological parameter.

When gastric constriction device 12 is implemented as a mechanical gastric band, the degree of constriction may be adjusted mechanically by means of a micro motor (not shown). The micro motor may be embedded within gastric constriction device 12 or controller 16. For example, a micro motor may be designed to adjust the degree of constriction provided by a mechanical gastric band, such as a telemetric adjustable gastric banding device. A telemetric adjustable gastric band device may enable an obstruction of the stoma to be removed without using an invasive procedure to deflate the band or endoscopy to remove the obstruction. Gastric constriction device 12 may also be any other type of mechanically adjustable gastric band. In any case, controller 16 includes circuitry designed to control the micro motor.

A gastric band used in constriction device 12 may be constructed in the form of a hollow tube that can be inserted through a laparoscopic cannula to completely encircle the upper end of the stomach and thus restrict the passage of food into the lower stomach. The gastric band generally may be fabricated from an elastomer, such as a medical grade silicone polymer or other suitable elastomer. In the example of FIG. 1, gastric constriction device 12 comprises a hollow tube having a first end, a second end, and a connection mechanism 15 that connects the first end and the second end such that gastric constriction device 12 forms a stoma opening in stomach 8. However, the illustrated example is merely exemplary and should not be considered limiting of the invention as broadly embodied and described in this disclosure.

Gastric constriction device 12 includes a plurality of electrodes 14 (not shown) for delivering electrical stimulation to patient 2. As will be described, one or more of electrodes 14 are selected to deliver electrical stimulation to patient 2 at a given time. In any case, electrodes 14 are integrally formed with gastric constriction device 12 such that electrodes 14 are positioned circumferentially around the restricted portion of stomach 8, e.g., with regular or irregular spacing. Specifically, electrodes 14 may be molded into gastric constriction device 12 such that each of electrodes 14 has at least a partially exposed surface that contacts patient 2 when gastric constriction device 12 is implanted within patient 2. Electrodes 14 may be integrally formed with gastric constriction device 12 using manufacturing techniques or processes similar to the techniques used to fabricate an implantable lead carrying a plurality of electrodes.

Electrodes 14 are electrically coupled to controller 16 implanted within patient 2. Controller 16 generates electrical stimulation pulses and lead 17 carries the electrical stimulation pulses to electrodes 14, i.e., electrodes 14 are electrically coupled to controller 16 via lead 17. For purposes of illustration, only a single lead is shown in FIG. 1. However, one or more leads may carry the electrical stimulation pulses to electrodes 14. Lead 17 carries a plurality of electrical conductors. Each of the conductors is electrically coupled, at one end, to a switch device such as a switch matrix within controller 16 and, at the other end, to one of electrodes 14. More specifically, controller 16 may include a pulse generator to generate electrical stimulation in the form of pulses and a switch device to select one or more of electrodes 14 to deliver the pulses to patient 2, as will be described in detail.

Accordingly, controller 16 includes, in addition to a control unit, a pulse generator that generates electrical stimulation pulses and a switch matrix that selects one or more of electrodes 14 and couples the electrical stimulation pulses to the selected electrodes to deliver the stimulation pulses to patient 2. Controller 16 includes a housing that encloses the control unit, e.g., a motor or a pump unit, the pulse generator, and the switch device.

The housing of controller 16 may be constructed with a biocompatible housing, such as titanium, stainless steel, silicone, or a polymeric material, and is surgically implanted within patient 2. The implantation site for controller 16 may be a subcutaneous location in the side of the lower abdomen or the side of the lower back. Lead 17 is flexible, electrically insulated from body tissues, and terminated with electrodes 14 integrally formed within gastric constriction device 12. In the illustrated example of FIG. 1, controller 16 is in fluid communication with gastric constriction device 12 via conduit 18. Conduit 18 may comprise a flexible interconnect member, such as a catheter or tube, that enables the transfer of fluid between controller 16 and, more specifically, the control unit within the housing of controller 16, and constriction device 12.

Because the circuitry associated with the control unit, pulse generator, and switch matrix are contained within the housing of controller 16, the circuitry may be integrated on a common circuit board. In particular, the control unit, pulse generator, and switch matrix may share one or more components of the circuitry, such as a processor and a memory. Consequently, the common circuit board may have less area than separate circuit boards for a control unit and a pulse generator and associated switch matrix. As a result, controller 16 may require less space within patient 2 and fewer incisions to implant thereby reducing the trauma experienced by patient 2 during the implantation procedure. Additionally, the cost of controller 16 may be less than the cost of purchasing a control unit separately from a pulse generator and associated switch matrix.

Controller 16 generates electrical stimulation pulses in accordance with a set of stimulation parameters. Thus, electrical stimulation pulses are characterized by stimulation parameters, such as voltage or current amplitude, pulse rate, pulse width, and electrode polarity. The stimulation parameters may be selected to suppress appetite in patient 2, e.g., by inducing a sensation of fullness or nausea. Alternatively or additionally, the stimulation pulses may be generated by IPG 16 to vary gastric motility. In one example, the stimulation pulses generated by IPG 16 may be selected to increase gastro intestinal motility. In particular, the stimulation pulses may cause the smooth muscle of duodenum and small intestine to contract and move contents toward the colon at an increased rate. In another example, the stimulation pulses generated by controller 16 may be configured to delay gastric emptying, e.g., by preventing the smooth muscle of stomach 8, such as the antrum, to contract or by disrupting the coordination of smooth muscle contraction and move contents from the entrance toward the exit of stomach 8. A combination of electrical stimulation to increase gastric motility in one region of the gastrointestinal tract and decrease gastric motility in a different region of the gastrointestinal tract may also be used.

Controller 16 selects one or more of electrodes 14 as an electrode combination to deliver the electrical stimulation pulses to patient 2. An electrode combination refers to the subset of electrodes selected from electrodes 14 and the polarities of the selected electrodes. An electrode combination may form one or more pairs of bipolar or multipolar electrode arrays. Alternatively, controller 16 may carry a reference electrode to form an “active can“arrangement in which electrodes 14 are unipolar electrodes referenced to the electrode on controller 16. Thus, a variety of polarities and electrode arrangements may be used.

For example, an electrode combination may include every other one of electrodes 14, i.e., a staggered or alternating configuration. Such an electrode combination enables electrical stimulation to be evenly delivered around the restricted portion of stomach 8. Alternatively, an electrode combination may include a number of adjacent electrodes thereby enabling electrical stimulation to be delivered to a localized region. In this case, the electrode combination may be selected to stimulate a nerve adjacent to the restricted portion of stomach 8, such as the vagus nerve or nerves that cause stomach 8 to contract and move food through stomach 8.

In addition, an electrode combination may deliver electrical stimulation in a variety of different modes, such as a continuous mode, in a series of bursts, or a combination of both. In some cases, rather than continuously delivering electrical stimulation over the course of a day, electrical stimulation may only be delivered over specific time intervals during the day. For example, electrical stimulation may be delivered in coordination with a specific event, such as during meal times or a sensed physiologic event. Electrical stimulation may, however, be delivered in a variety of different modes over a specific time period. In some cases, electrical stimulation may be suspended during times at which the patient is sleeping. Alternatively, stimulation may be delivered on a full-time basis.

More than one electrode combination may deliver electrical stimulation to patient 2. In such embodiments, a first electrode combination may deliver electrical stimulation in accordance with a first set of stimulation parameters and a second electrode combination may deliver electrical stimulation in accordance with a second set of stimulation parameters. The first and second electrode combinations may deliver electrical stimulation at the same time or on a time-interleaved basis. For time-interleaved delivery, stimulation pulses may be delivered in an overlapping or non-overlapping manner, such that stimulation pulses delivered to different selected electrode sets are delivered in respective overlapping or non-overlapping time slots. In any case, the effect resulting from electrical stimulation, i.e., suppressing the appetite of a patient or varying, i.e., increasing or decreasing, gastric motility, depends on the positions and polarities of the electrodes and the parameters associated with the stimulation pulses.

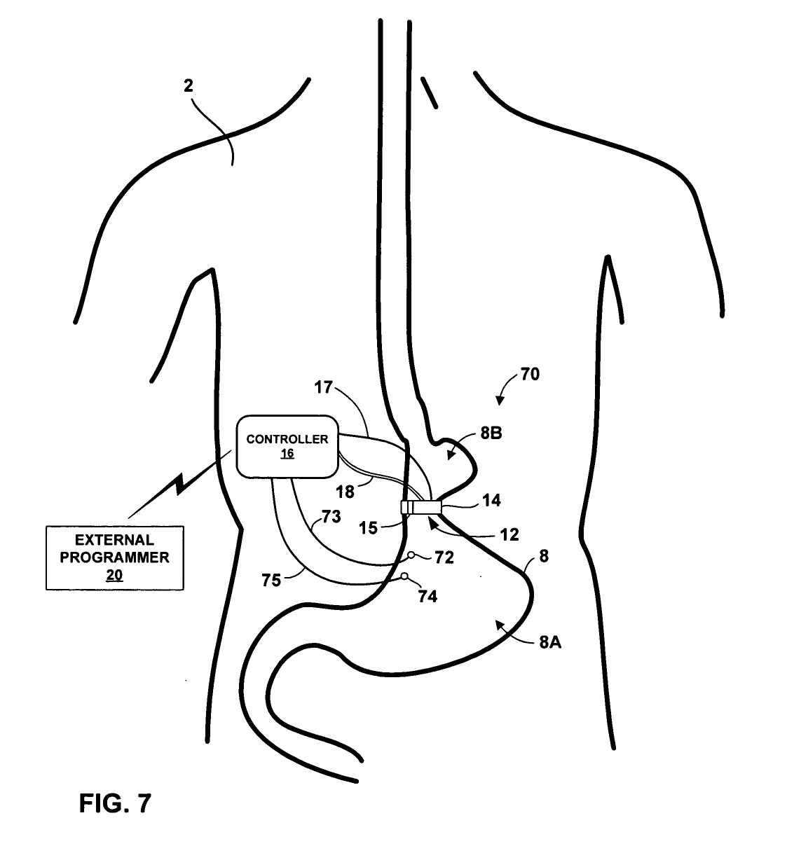

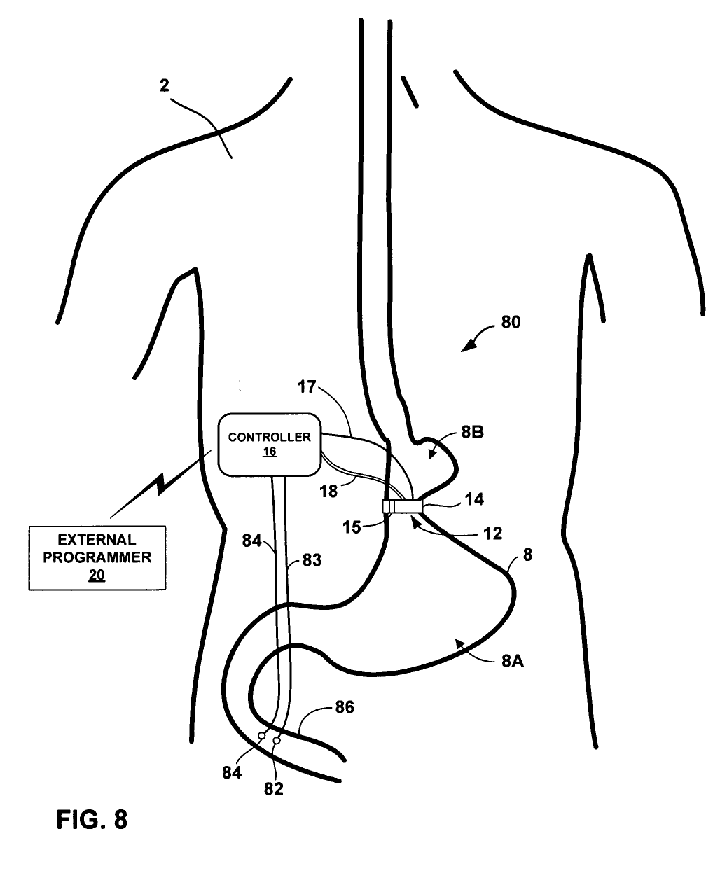

In some embodiments, however, electrical stimulation pulses may be delivered to other areas within the gastrointestinal tract, such as the upper stomach, lower stomach, esophagus, duodenum, small intestine, or large intestine, in addition to the restricted portion of stomach 8. In such embodiments, electrodes (not shown) may be implanted at the target organ/location and coupled to implantable stimulation via corresponding leads (not shown). For example, FIGS. 7 and 8 illustrate electrodes implanted at the stomach and duodenum, respectively, in combination with system 10. Hence, controller 16 may be coupled to deliver stimulation energy within gastric constriction device 12 as well as electrodes outside of constriction device 12. Delivering electrical stimulation at other areas within the gastrointestinal tract may further enhance or delay gastric motility or suppress the appetite of the patient.

A clinician may test all or at least a portion of the possible electrode combinations of electrodes within the plurality of electrodes in order to identify an effective combination of electrodes and their polarities. Efficacy may be judged in terms of therapeutic effect in suppressing appetite, reducing food intake (liquid or solid), or by modifying (increasing or decreasing) gastric motility, gastrointestinal myoelectric activity, and in terms of the absence of undesirable side effects. Undesirable side effects may be evaluated by monitoring heart rate variability, changes in plasma hormone levels, and brain imaging. Efficacy also may be judged in terms of power efficiency provided by the selected electrode combination, particularly in light of the limited battery resources that may be available within an IPG.

The process of selecting values for the stimulation parameters that provide adequate results may be time consuming and require substantial trial and error before an effective program is identified. A clinician may need to test all possible electrode combinations or a significant portion thereof in order to identify an effective electrode combination. Consequently, in some cases, the clinician may test electrode combinations by manually specifying each combination to test based on intuition or some idiosyncratic methodology, and recording notes on the efficacy and side effects of each electrode combination after delivery in order to later compare and select from the tested electrode combinations.

The magnitude of such a task may quickly become too time consuming and costly as the number of electrodes 14 integrally formed with gastric constriction device 12 increases. Accordingly, controller 16 may utilize a search algorithm to select electrode combinations to test. Controller 16 may receive input from the patient to indicate preferred electrode combinations. For example, patient 2 may enter input to external programmer 20 in wireless communication with controller 16. Controller 16 may store electrode combinations in internal memory in response to receiving input from the patient. The electrode combinations may be stored as programs in combination with stimulation parameters such as voltage or current amplitude, stimulation pulse width, and pulse rate.

Controller 16 may also include telemetry electronics to communicate with external programmer 20. External programmer 20 may be a small, battery-powered, portable device that accompanies patient 2 throughout a daily routine. External programmer 20 may have a simple user interface, such as a button or keypad, and a display or lights. External programmer 20 may be a hand-held device configured to permit activation of stimulation, selection of electrode combinations or stimulation programs, and adjustment of stimulation parameters. The stimulation parameters may be fixed or adjusted in response to patient input entered via external programmer 20. For example, in some embodiments, patient 2 may be permitted to adjust stimulation amplitude and turn stimulation on and off. Alternatively, programmer 20 may form part of a larger device including a more complete set of programming features including complete parameter modifications, firmware upgrades, data recovery, or battery recharging in the event controller 16 includes a rechargeable battery.

External programmer 20 may also be configured to enable a clinician or patient to control the degree of constriction of gastric constriction device 12 and retrieve information stored in controller 16. Typically, only a clinician may be permitted to change the degree of gastric constriction of gastric constriction device 12, although adjustment by a patient may be permitted in some circumstances. During an office visit, a clinician may download data stored in controller 16 to external programmer 20. The clinician may view the information thereby allowing the physician to assess the course of treatment and determine whether any adjustments are necessary. For example, the clinician may view data indicative of the degree of gastric constriction and determine if an adjustment is necessary. When an adjustment is desired, the clinician may program controller 16 to reduce the degree of gastric constriction, i.e., cause the surface of gastric constriction device 12 to be tightened or loosened, using external programmer 20.

Various surgical procedures may be used for implanting system 10 within patient 2. Well known open surgical procedures or laparoscopic surgical procedures for implanting gastric banding devices may be used to implant gastric constriction device 12 and controller 16 within patient 2. Generally, a surgeon may first implant gastric constriction device 12. The surgeon may then implant controller 16 and couple controller 16 to gastric constriction device 12, e.g., by connecting conduit 18 and lead 17 to respective inputs or connectors on gastric constriction device 12. By enclosing control circuitry and other components for controlling the degree of gastric constriction applied by gastric constriction device and the delivery of electrical stimulation within a common housing, the size of controller 16 may be reduced. Thus, patient 2 may experience less trauma as a result of the surgical procedures to implant system 10 and, more particular, controller 16.

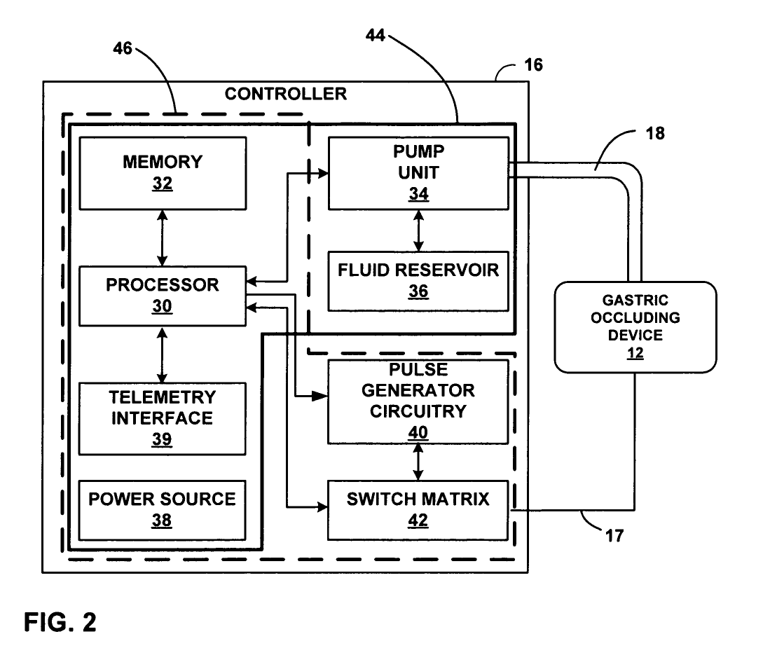

FIG. 2 is a block diagram illustrating controller 16. As described above, controller 16 includes a control unit, a pulse generator, and a switch matrix. The control unit hydraulically actuates gastric constriction device 12 by injecting or withdrawing fluid to and from gastric constriction device 12. The pulse generator generates electrical stimulation pulses and the switch matrix selects one or more of electrodes 14 and couples the electrical stimulation pulses to the selected electrodes to deliver the stimulation pulses to patient 2. In the illustrated example of FIG. 2, control unit 44 includes pump unit 34, fluid reservoir 36, processor 30, memory 32, power source, 38, and telemetry interface 39. FIG. 2 also illustrates pulse generator 46 which includes circuitry that operates as the previously described pulse generator and switch matrix. As shown in FIG. 2, pulse generator 46 includes pulse generator circuitry 40, switch matrix 42, processor 30, memory 32, power source 38, and telemetry interface 39. Consequently, control unit 44 and pulse generator 46 share processor 30, memory 32, power source 38, and telemetry interface 39. However, FIG. 2 is merely exemplary and should not be considered limiting of the invention as broadly embodied and described in this disclosure.

In some cases, control unit 44 and pulse generator 46 share at least a portion of the circuitry or electrical components within controller 16. By sharing at least a portion of the electrical components, e.g., a processor, memory, telemetry interface, power source, telemetry interface, and other commonly used electrical components, the size of the circuit board on which the electrical components are fabricated may be reduced. The reduced size of controller 16 may achieve particular advantages, such as reducing the trauma experienced by patient 2 during and after implantation and reducing cost.

Accordingly, processor 30 may store instructions for controlling the degree of gastric constriction provided by gastric constriction device 12. Processor 30 may take the form of a microprocessor, digital signal processor (DSP), application specific integrated circuit (ASIC), field-programmable gate array (FPGA), or other logic circuitry. Pump unit 34 operates under the control of processor 30 to adjust the degree of gastric constriction provided by constriction device 12 by injecting or withdrawing fluid to and from constriction device 12. Fluid reservoir 36 contains a fluid, such as saline or another fluid, that may be injected to or withdrawn from gastric constriction device 12 to control the degree of gastric constriction. Fluid reservoir 36 may provide access for filling, e.g., by percutaneous injection of fluid via a self-sealing injection port.

Pump unit 34 pumps the fluid from fluid reservoir 36 and injects the fluid into an expandable lumen of gastric constriction device 12 thereby decreasing the inner diameter of device 12 and increasing the degree of gastric restriction. Pump unit 34 is in fluid communication with gastric constriction device 12 via conduit 18. Conduit 18 may comprise a flexible interconnect member, such as a catheter, that enables the transfer of the fluid between pump unit 34 and device 12. In addition, pump unit 34 can withdraw fluid from gastric constriction device 12 back to fluid reservoir 36 thereby increasing the inner diameter of device 12 and decreasing the degree of gastric restriction.

Memory 32 stores instructions that may be executed by processor 30 to control the degree of gastric constriction provided by gastric constriction device 12. Memory 32 may include a read-only memory (ROM), random access memory (RAM), electronically-erasable programmable ROM (EEPROM), flash memory, or the like. Memory 32 stores instructions that may be executed by processor 30 and thereby control the degree of gastric constriction of gastric constriction device 12. For example, processor 30 may also store data collected during treatment and/or monitoring of patient 14 within memory 32.

Memory 32 may store a schedule of times for adjusting the degree of gastric constriction and values for various degrees of gastric constriction. Processor 30 executes the instructions to cause pump unit 34 to adjust the degree of gastric constriction provided by device 12. In some embodiments, processor 30 may vary the amount of constriction over the course of a day, or adjust constriction at particular time periods of the day. As an example, in some embodiments, processor 30 may cause pump unit 34 to decrease gastric constriction during preset meal times in order to allow the patient to ingest food. Processor 30 causes pump unit 34 to increase the degree of gastric constriction when it is not a preset meal time in order to limit ingestion of food by the patient. Preset meal times and values that determine the degree of constriction are stored in memory 32 and accessed by processor 30.

Processor 30 may also store data collected during treatment and/or monitoring of a patient within memory 32. For example, in some embodiments, system 10 may include pressure sensors that generate an electrical signal indicative of the degree of gastric constriction provided by gastric constriction device 12. System 10 may also include sensors for sensing a physiological parameter. The sensors may be incorporated with gastric constriction device 12 or separate from device 12. In either case, processor 30 receives the signal generated by the sensor(s) and, based on the signal, controls pump unit 34 accordingly. In particular, processor 30 processes and analyzes the received signal to determine if the degree of gastric constriction needs to be adjusted. If gastric constriction needs to be adjusted, processor 30 determines the amount that the gastric constriction should be adjusted.

Although control unit 44 is described as hydraulically operating gastric constriction device 12, control unit 44 may also mechanically operate gastric constriction device 12. In such embodiments, control unit 44 may include a micro motor that mechanically increases and decreases the inner diameter of gastric constriction device 12 to control the degree of gastric constriction instead of pump unit 34 and fluid reservoir 36. Such a motor may wind and unwind a belt or other elongated member to tighten or loosen device 12. Therefore, control unit 44 as shown in FIG. 2 should not be considered limiting to the invention as broadly embodied and described in this disclosure. Rather, control unit 44 may comprise any control electronics and devices that control the functioning, i.e., degree of gastric constriction, of a gastric constriction device.

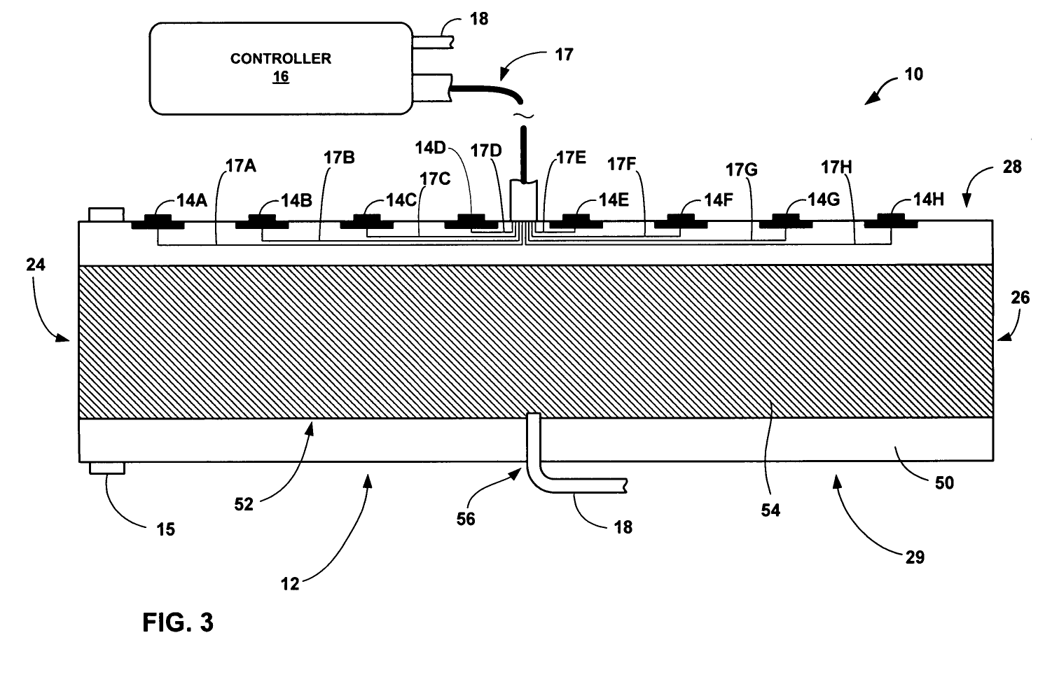

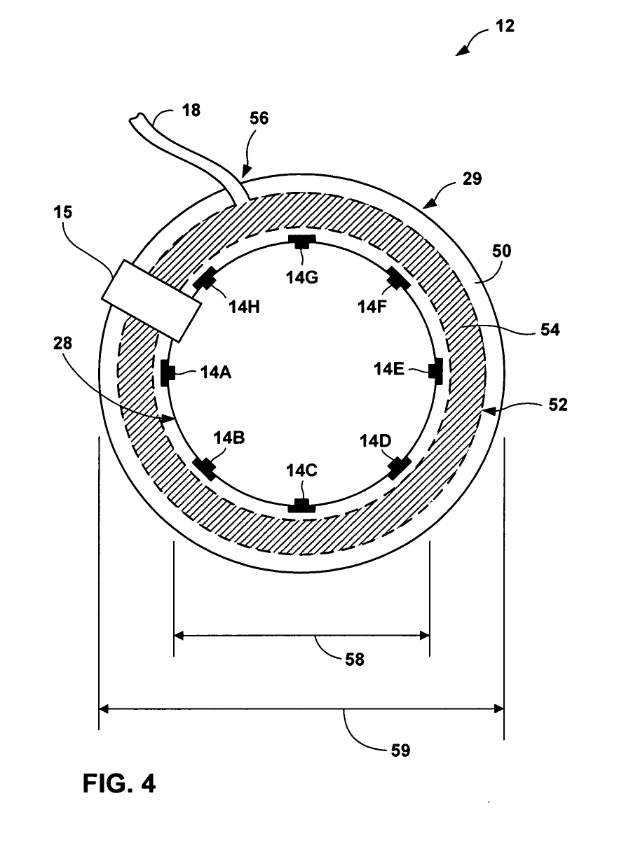

Pulse generator 46 controls the delivery of electrical stimulation to the patient via electrodes 14 integrally formed with gastric constriction device 12. As described above, electrodes 14 are positioned circumferentially around the restricted portion of stomach 8 with even or irregular spacing and deliver electrical stimulation to limit food intake and vary gastric motility. Electrodes 14 are electrically coupled to pulse generator 46 via lead 17, which may include a separate lead conductor for each of electrodes 14 or a bundle of conductors. In general, electrodes 14 may include any number and type of electrodes. However, although eight electrodes are shown in FIGS. 3 and 4, a greater or lesser number of electrodes may be integrally formed with gastric constriction device 12 to deliver stimulation to patient 2. FIGS. 4B-4D illustrate various configurations with different numbers of electrodes and patterns of electrodes.

In general, a relatively large number of electrodes, e.g., from eight to thirty-two, may be desirable in order to permit selection of a greater number of bipolar, multipolar, and unipolar electrode combinations to deliver electrical stimulation. The availability of multiple, selectable electrode combinations increases the probability that an efficacious electrode combination will be found. In particular, a larger array of electrodes extending around the stomach permits delivery of stimulation energy to a variety of target stimulation sites on a selective basis, or delivery of stimulation energy to multiple target stimulation sites either simultaneously or on a time-interleaved basis.

As shown in FIG. 2, pulse generator 46 includes a processor 30, memory 32, pulse generator circuitry 40, switch matrix 42, power source 38, and telemetry circuitry 39. Memory 32 stores instructions for execution by processor 30 and stimulation parameters, such as voltage and current amplitude, pulse width, and pulse rate. Memory 32 may also record stimulation therapy data for long term storage and retrieval by patient 2 or a clinician. For example, memory 32 may store preferred electrode combinations and stimulation parameters. Alternatively, stored stimulation therapy data may be used in the adjustment of stimulation parameters. Memory 32 may include a single memory or separate memories for storing instructions, stimulation parameters sets, stimulation information, and information used by control unit 44.

Processor 30 controls pulse generator circuitry 40 to deliver electrical stimulation to patient 2 in addition to controlling pump unit 34. Based on stimulation parameters stored in memory 32 or programmed by external programmer 20, processor 30 controls pulse generator circuitry 40 and switch matrix 42 to deliver appropriate stimulation. As described above, processor 30 may instruct pulse generator circuitry 40 to generate electrical stimulation in accordance with various modes, e.g., continuously, in a series of bursts, or a combination of both. Additionally, each pulse may be delivered in accordance with a different set of stimulation parameters. Again, processor 30 may take the form of a microprocessor, DSP, ASIC, FPGA, or other equivalent integrated or discrete logic circuitry.

Pulse generator circuitry 40 comprises circuits, such as capacitors and switches, for the generation of electrical stimulation in the form of pulses. Pulse generator circuitry 40 may deliver the pulses to switch matrix 42, which comprises an array of switches. Processor 30 interacts with switch matrix 42 to select one or more electrodes for delivery of generated stimulation pulses. As previously described, processor 30 may select one or more of electrodes 14 and the polarities of each of the selected electrodes, i.e., an electrode combination, to deliver electrical stimulation to the patient. In some embodiments, processor 30 may select more than one electrode combination. In such embodiments, each electrode combination may deliver electrical stimulation in accordance with a different set of stimulation parameters. Additionally, the electrode combinations may deliver electrical stimulation at the same time or on a time-interleaved basis. In any case, based on the selected electrode combinations made by processor 30, switch matrix 42 delivers the pulses to the to the selected electrodes via wires of lead 17 that are electrically connect the electrodes to pulse generator 46.

As a further alternative, the electrode combinations may be selected so that stimulation rotates or revolves about the gastric band that encircles stomach 8 by sequentially activating selected electrode combinations. As an illustration, if there are eight electrodes (E0 through E7) arranged linearly around the inner surface of the gastric band, controller 16 may sequentially activate bipolar pairs of electrodes in the following order: E0-E1, E1 -E2, E2-E3, E3-E4, E4-E5, E5-E6, E6-E7. The time between activation of successive electrode pairs may be adjusted to achieve different transition rates between the electrodes.

In general, by sequentially activating electrodes that are physically positioned in a linear array around the gastric band, stimulation energy can be made to move around the constricted portion of the gastrointestinal tract. Stimulation can be made to move around the entire constricted portion or only a segment of the circumference of the constricted portion. In addition, stimulation may proceed around the circumference in repeated orbits in one direction, or complete one orbit or a partial orbit, and then reverse direction. Reversal of orbit direction may occur on a repetitive basis. Arrangement of electrodes on the gastric band permits controller 16 to target particular stimulation sites, access multiple stimulation sites on a continuous or time-interleaved basis, or access multiple stimulation sites in sequence.

Control unit 44, pulse generator 46, or both may include telemetry circuitry 39, which enables processor 30 to communicate with external programmer 20 other external devices, via RF telemetry, proximal inductive interaction of controller 16 with external programmer 20, or other type of wireless communication. Processor 30 controls telemetry circuitry 39 to exchange information, e.g., operational information, with external programmer 20. As an example, external programmer 20 and controller 16 may be configured to enable a clinician or patient to turn stimulation on and off or adjust stimulation amplitude or intensity using external programmer 20. However, patient 2 may not be permitted to adjust the degree of gastric constriction applied by gastric constriction device 12. Processor 30 may also transmit operational information to external programmer 20 via telemetry circuitry 39 thereby allowing a clinician to view the course of treatment and determine if adjustments are necessary.

The illustrated components of controller 16, i.e., the components of control unit 44 and pulse generator 46, receive energy from power source 38, such as a battery or other suitable power source. In some embodiments, power source 38 is rechargeable and power source 38 receives energy inductively captured by a recharge module (not shown). Power management circuitry (not shown) may control the recharging and discharging of power source 38. In other embodiments, power source 38 includes a nonrechargeable battery. In additional embodiments, power source 38 may receive operating power by inductive energy transfer with an external power source.

FIG. 3 is a lengthwise cross-sectional side view of gastric constriction device 12 of FIG. 1. In particular, FIG. 2 illustrates gastric constriction device 12 prior to implantation within a patient. Band 50 of gastric constriction device 12 includes an expandable lumen 52 extending longitudinally from a first end 24 to a second end 26 of band 50. When implanted within a patient, first end 24 and second end 26 are connected together via connection mechanism 15 to encircle and partition a portion of a patient's gastrointestinal tract thereby restricting ingestion of food by the patient. FIG. 3 illustrates gastric constriction device 12 connected in this manner.

In use, expandable lumen 52 is at least partially filled with a fluid 54 to restrict a portion of a patient's gastrointestinal tract. The degree of gastric constriction depends on the amount of fluid 54, e.g., saline or another fluid, within band 50 and, more particularly, lumen 52. Controller 16 and, more particularly, control unit 44 within controller 16, includes a fluid reservoir (not shown) and a pump unit (not shown) that pumps fluid 54 from the reservoir through conduit 18 to gastric constriction device 12. As shown in FIG. 2, controller 16 is in fluid communication with lumen 52 via conduit 18 which enters lumen 52 through an aperture 56 in band 50. The pump unit may also pump fluid 54 from lumen 52 back to the reservoir to enlarge the size of the stoma opening.

In some cases, controller 16 may control the degree of gastric constriction in response to input received from external programmer 20 (FIG. 1). Alternatively, controller 16 may receive input from one or more sensors (not shown) implanted within the patient and control the degree of gastric constriction based on the input. For example, controller 16 may adjust the degree of gastric constriction in response to a sensed physiological event, such as ingestion of food. In a further embodiment, controller 16 may adjust the degree of gastric constriction over particular time periods during the course of a day. For example, controller 16 may increase the degree of gastric constriction by pumping fluid 54 from a fluid reservoir into lumen 52 during meal times and decrease the degree of gastric constriction by pumping fluid 54 from lumen 52 back into the reservoir at night and during the time periods between meals. Control unit 20 may also adjust the degree of gastric constriction to relieve obstruction of the stoma by food without using an invasive procedure to deflate the band or endoscopy to remove the obstruction.

Alternatively, controller 16 may include an injection port instead of a pump unit and a fluid reservoir. In such embodiments, fluid 54 is injected or withdrawn from lumen 52 by inserting a needle into controller 16. In such embodiments, controller 16 may be implanted just under the patient's skin. Thus, each time the degree of gastric constriction needs to be adjusted, the patient's skin must be punctured resulting in discomfort for the patient and an increased risk of infection. As a result, multiple adjustments to maintain the optimal degree of gastric constriction may be required thereby increasing the cost and number of medical visits.

In the illustrated example, electrodes 14A-H (collectively referred to as “electrodes 14”) are integrally formed with band 50 of gastric constriction device 12. In particular, each of electrodes 14 includes a portion integrally form with band 50 and an exposed surface that contacts the stomach when implanted within a patient. Electrodes 14 are electrically coupled to controller 16 via lead 17 containing electrical conductors 17A-17H, which are coupled to respective electrodes 14A-14H. In some embodiments, each of electrodes 14 may be coupled to controller 16 via a separate lead wire. However, bundling of conductors 17A-17H within a common lead 17 ordinarily will be more desirable. Conductors 17A-17H are embedded into the band 50 of gastric constriction device such that they do not contact fluid 54. For example, conductors 17A-17H may be electrically insulated and fluid sealed and/or reside within a wall of band 50, away from contact with fluid 54.

Electrodes 14 are integrally formed with band 50 such that electrodes 14 are positioned circumferentially around restricted portion of the patient's gastrointestinal tract with even spacing when implanted within the patient. Accordingly, electrodes 14 are positioned along the inner surface 28 of gastric constriction device 12 as shown in FIG. 2. By evenly spacing electrodes 14, controller 16 can select electrode combinations to evenly distribute electrical stimulation around the restricted portion of the patient's gastrointestinal tract. In addition, a group of adjacent electrodes can be selected to deliver electrical stimulation to a localized area of the restricted portion of the gastrointestinal tract.

Alternatively or additionally, a plurality of electrodes may be similarly positioned around the outer surface 29 of band 50. By positioning electrodes around outer surface 29, electrical stimulation may be delivered to nerves proximate to the stomach, but outside the stomach wall. Stimulation of nerves proximate to stomach 8 may further induce a feeling of fullness or nausea to suppress the appetite of the patient or cause muscle of the stomach to contract and move food from the entrance of the stomach to the exit thereby enhancing gastric motility and reducing caloric absorption. As lumen 52 expands and contracts to increase or decrease the inner surface of gastric constriction device 12, the position of electrodes 14 may shift accordingly so that the electrodes remain evenly spaced.

In FIG. 3, gastric constriction device 12 includes eight electrodes, i.e., electrodes 14, integrally formed with band 50 for purposes of illustration. However, gastric constriction device 12 may include a lesser or greater number of electrodes. A gastric constriction device having numerous electrodes may be particularly advantageous because the number of possible electrode combinations increases with the number of electrodes integrally formed with gastric constriction device. In other words, providing a large number of electrode combinations increases the likelihood of discovering an electrode combination that achieves a high clinical efficacy with minimal side effects experienced and favorable power consumption characteristics.

Controller 16 includes a switch device for selecting one or more electrodes or electrode combinations to deliver electrical stimulation to the patient as previously described in FIG. 1. For example, a selected electrode combination may deliver electrical stimulation in accordance with various modes, e.g., continuously, in a series of bursts, or a combination of both. The electrode combination may also deliver electrical stimulation according to different stimulation parameters at different times during the day. When more than one electrode combination delivers electrical stimulation, each selected electrode combination may deliver electrical stimulation in accordance with a different set of stimulation parameters. The electrode combinations may deliver electrical stimulation at the same time or on a time-interleaved basis.

Various surgical procedures may be used for implanting system 10 within patient 2. In some cases, controller 16 may be implanted using well known surgical techniques for implanting an implantable medical device within a subcutaneous pocket of the lower abdomen or lower back of a patient. With respect to gastric constriction device 12, the surgeon may implant device 12 to constrict stomach 8 as shown in FIG. 1. In particular, first and second ends 24, 26 are connected together via connection mechanism 15 to form a ring configuration, that partitions the gastrointestinal tract into an upper and lower region (FIG. 4). Connection mechanism 15 may be any type of fastening mechanism adapted to attach the two ends of band 50 together. Connection mechanism 15 may include, for example, a buckle, sutures, a clamp, adhesive, surgical staples, a coupling, or any other type of biocompatible fastener. In some embodiments, connection mechanism 15 may be a tab that interfaces with a slot or hole in the opposite end of band 50. The surgeon may couple controller 16 to gastric constriction device 12, e.g., by connecting conduit 18 and lead 17 to respective inputs or connectors on gastric constriction device 12.

Although a hydraulic banding device is shown in FIG. 3, gastric constriction device 12 may alternatively comprise a mechanical gastric constriction device or other type of gastric constriction device. The purpose of FIG. 3 is to illustrate the manner in which electrodes 14 are integrally formed with band 50 of gastric constriction device 12. Accordingly, FIG. 3 is merely exemplary and should not be considered limiting of the invention as broadly embodied and described in this disclosure.

FIG. 4 illustrates an example configuration of electrodes 14 integrally formed with band 50 when implanted within a patient. Accordingly, electrodes 14 are positioned circumferentially along inner surface 28 with even spacing. Each of electrodes 14 has a portion integrally formed with band 50 and an exposed portion which contacts the stomach (not shown) when gastric constriction device 12 is implanted to restrict food intake of patient 2. As lumen 52 expands to decrease inner diameter 58 (increase gastric constriction) and relaxes to increase inner diameter 58 (decrease gastric constriction), electrodes 14 move accordingly. In general, in embodiments where electrodes 14 are regularly spaced, electrode 14 may remain equally spaced as the degree of gastric constriction is adjusted by controller 16.

Inner surface 28 may expand more easily than outer surface 29 so that inner diameter 58 can be controlled more precisely. This may be achieved by forming inner surface 28 and outer surface 29 from different materials. In this case, band 50 may be made of an inner wall and an outer wall joined together by heat-sealing, gluing, solvent bonding, or mechanical means such as suturing or riveting. Thus, the inner wall and outer wall are joined to form an expandable cavity in which the outer wall expands to a substantially lesser degree than the inner wall.

As previously described, electrodes may be positioned along outer surface 29 in addition to or in place of electrodes 14. In either case, the electrodes may be positioned in a similar fashion as electrodes 14 along inner surface 28. Integrally forming electrodes along outer surface 29 may be particularly advantageous in embodiments in which outer surface is formed from a substantially non-expansible material thereby enabling the electrodes to deliver electrical stimulation to the same target area regardless of the degree of constriction of gastric banding device 12. However, electrodes integrally formed with outer surface 29 may generally be beneficial by delivering electrical stimulation to nerves proximate to the stomach wall or gastrointestinal tract of a patient.

For ease of illustration, not all of the components of gastric constriction device 12 and system 10 are shown in FIG. 4. For example, although conduit 18 is shown entering lumen 52 via aperture 56 in band 50, controller 16 is not shown. In addition, controller 16 and lead 17, which electrically couples controller 16 to electrodes 14A-14H via conductor 17A-17H, respectively, are not shown. Accordingly, FIG. 4 is merely illustrative and should not be considered limiting of the invention as broadly embodied and described within this disclosure.

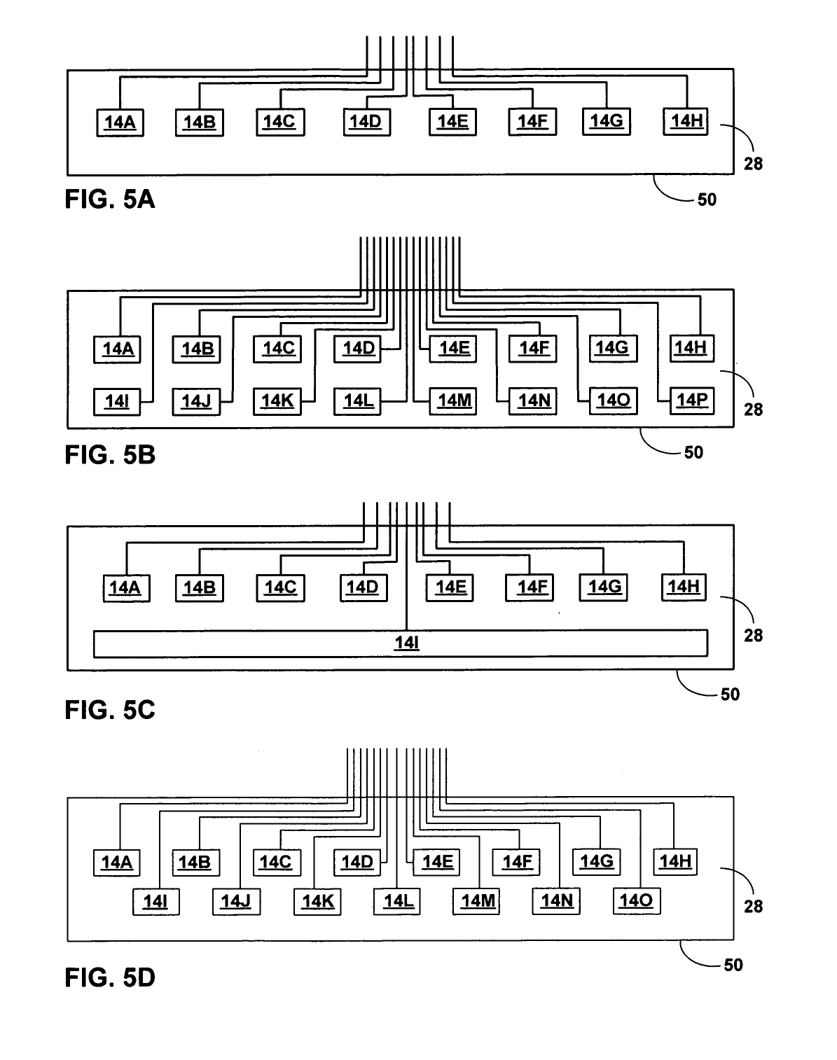

FIGS. 5A-5D are plan views of an interior side, i.e., inner surface 28, of a gastric constriction device in the form of gastric band 50 of FIG. 3, illustrating various example electrode patterns. FIG. 5A shows a linear array of electrodes 14A-14H that extend along the length of gastric band 50. In the example of FIG. 5A, electrodes 14A-14H are arrange along a common axis parallel to a longitudinal axis of band 50. Electrodes 14A-14H may be selected to form bipolar or multipolar electrode combinations. Alternatively, one electrode 14A-14H may be selected to form a unipolar combination with an electrode carried or formed by a housing of controller 16. In either case, by selectively using one or more electrodes 14A-14H, one or more stimulation sites may be selected at different positions along the length of gastric band 50, i.e., about the periphery of the portion of the stomach constricted by the gastric band.

In the example of FIG. 5B, gastric band 50 includes two linear arrays of electrodes 14A-14H and 14I-14P that extend parallel to one another along the length of the gastric band. In FIG. 5B, electrodes 14A-14H are substantially aligned with electrodes 14I-14P, respectively, along the length of gastric band 50. One or more electrodes 14A-14H, 14A-14P in one linear array may be selected in combination with one or more other electrodes in the same linear array, or with one or more electrodes in the other linear array, or with a common electrode carried or formed by a housing of controller 16. Although two linear arrays are shown in FIG. 5B, multiple linear arrays may be provided. In addition, such linear arrays may be arranged as multiple rows, as well as multiple columns, permitting row/column addressing to select electrodes for desired electrode combinations.

FIG. 5C shows a pattern of electrodes include a linear array of electrodes 14A-14H and a continuous electrode 14I that extends along a major portion of the length of gastric band 50. In the example of FIG. 5C, continuous electrode 14I may serve as a common electrode to form a bipolar or multipolar electrode combination with one or more of electrodes 14A-14H. In other embodiments, continuous electrode 14I may be used in combination with electrodes arranged in multiple linear arrays, e.g., on opposite sides of the continuous electrode.

In the example of FIG. 5D, gastric band 50 includes two linear arrays of electrodes 14A-14H and 14I-14P that extend parallel to one another along the length of the gastric band. In contrast to FIG. 5B, however, FIG. 5D shows the linear arrays arranged so that electrodes 14A-14H are not substantially aligned with electrodes 14I-14P, respectively, along the length of gastric band 50. Instead, electrodes 14A-14H, 14A-14P in one linear array are at staggered linear positions relative to electrodes in the other linear array. As in the example of FIG. 5B, consistent with FIG. 5D, multiple (e.g., two or more) linear arrays of electrodes may be provided in gastric band 50.

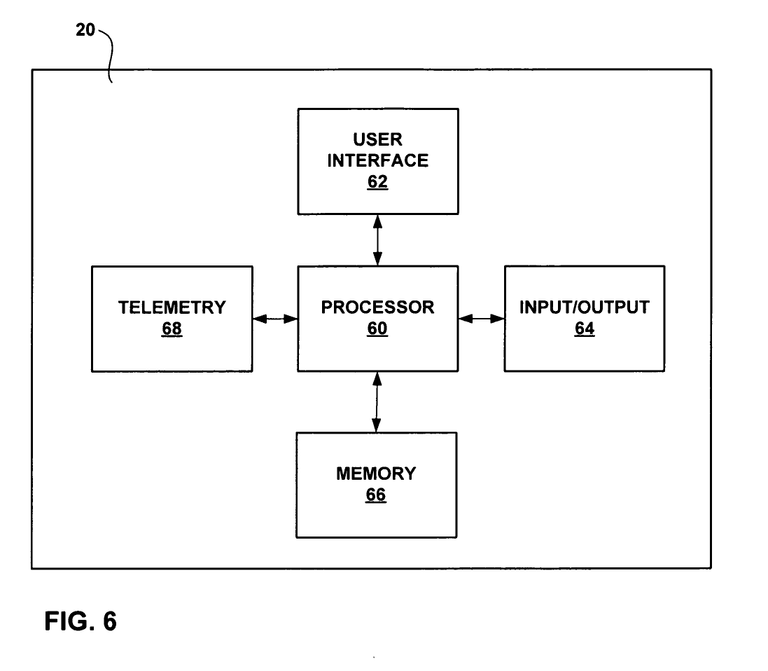

FIG. 6 is a block diagram illustrating an example of external programmer 20 in wireless communication with gastric constriction device 12. In general, external programmer 20 allows a user, such as a patient or clinician, to program delivery of electrical stimulation, program or control the degree of gastric constriction provided by device 12, or both.

External programmer 20 may be a small, battery-powered, portable device that accompanies patient 2 throughout a daily routine. User interface 62 may include a simple user interface, such as a button or keypad, and a display or lights. Processor 60 may also provide a graphical user interface (GUI) to facilitate interaction with the user, as will be described in detail. Processor 60 may include a microprocessor, a controller, a DSP, an ASIC, an FPGA, or other control circuitry.

External programmer 20 also includes a memory 66 that may store sets of stimulation parameters including selected electrode combinations, values for adjusting the degree of gastric constriction, and schedules for delivering electrical stimulation and adjusting the degree of gastric constriction at respective times. Generally, stored information may be available only to a clinician or other authorized user. In this manner, a clinician may program delivery of electrical stimulation by specifying parameter sets and control the degree of gastric constriction by specifying values, such as the inner diameter of gastric constriction device 12. In some cases, however, patient 2 may be permitted to adjust stimulation amplitude and/or constriction degree, and turn stimulation and/or constriction on and off.

Processor 60 transmits the selected electrode combinations, sets of stimulation parameters for deliver electrical stimulation via the selected electrode combinations, and values for adjusting the degree of gastric constriction to controller 16. Processor 60 transmits the information via wireless telemetry circuitry 68. Processor 60 also includes input/output circuitry 64 for transmitting and receiving information over a wired connection or removable electrical, magnetic, or optical media, e.g., to exchange information with another programming device.

External programmer 20 may be configured to store sets of stimulation programs and program groups, and download such programs and program groups to controller 16 when a change is requested. Alternatively, controller 16 may store complete sets of stimulation programs and program groups, in which case external programmer 20 downloads instructions for selection of one or more programs or programs groups stored in controller 16.

In general, the term “program” may refer to a combination of parameter settings, including one or more of electrode combination, electrode polarity, pulse amplitude (current or voltage), pulse width and pulse rate, used to provide stimulation therapy. A program of stimulation therapy may be delivered alone or in combination with other programs, e.g., simultaneously via multiple stimulation channels or on a time-interleaved basis via one or more stimulation channels.

The term “group,” as used in this disclosure, may generally refer to a therapeutic stimulation therapy including one or more programs. For example, the programs in a group may be delivered, as described above, simultaneously or on a time-interleaved basis. In other words, the programs in a group of programs are delivered together in combination with one another.

FIG. 7 is a schematic diagram illustrating an example implantable system 70 configured for the treatment of obesity. Implantable system 70 includes components similar or identical to the components of system 10, but further includes electrodes 72 and 74 coupled to controller 16 via leads 73 and 75, respectively. The components that are shared or, more specifically, common to system 10 and system 70 are identified by the same numbering in FIGS. I and 7. Accordingly, system 70 operates and performs in a similar fashion as system 10 but with added stimulation features because of additional electrodes 72 and 74.

In particular, by delivering electrical stimulation to lower stomach 8B via electrodes 72, 74, in addition to delivery of stimulation to the restricted portion of stomach 8 via electrodes 14 in combination with gastric banding, system 70 may more completely address or treat the factors contributing to obesity. For example, the additional electrical stimulation delivered by electrodes 72 and 74 may be selected to enhance the sensation of fullness or nausea to limit ingestion of food by patient 2 or vary gastric motility, i.e., enhance gastric motility to reduce caloric absorption from the ingested food beyond that which can be achieved by system 10, or delay gastric emptying.

In the illustrated example, leads 73 and 75 terminate into tissue of lower stomach 8B at electrodes 72 and 74, respectively. Electrodes 72 and 74 may comprise any number and type of electrodes, such as conventional ring electrode leads, paddle electrode leads, and other electrodes suitable for delivering electrical stimulation to lower stomach 8B. The stimulation pulses generated by controller 16 cause the smooth muscle of lower stomach 8B to contract and slowly move the contents from upper stomach 8A toward the exit of lower stomach 8B. Alternatively or additionally, the electrical stimulation pulses may stimulation nerves within lower stomach 8B to cause muscle contraction and thereby enhance gastric motility.

The electrodes carried at the distal end of each of leads 73 and 75 may be attached to the wall of lower stomach 8B in a variety of ways. For example, electrodes 72 and 74 may be surgically sutured onto the outer wall of lower stomach 8B or fixed by penetration or anchoring devices, such as hooks, barbs, or helical structures within the tissue of lower stomach 8B. Surgical adhesives may also be used to attach electrodes 72 and 74 to lower stomach 8B. In any case, electrodes 72 and 74 are implanted in acceptable electrical contact with the smooth muscle cells within the wall of lower stomach 8B. In some embodiments, electrodes 72 and 74 may be placed on the serosal surface of lower stomach 8B, within the muscle wall of stomach 8B, or within the mucosal or submucosal region of lower stomach 8B.

FIG. 8 is a schematic diagram illustrating an example implantable system 80 configured for the treatment of obesity. Similar to implantable system 70, implantable system 80, as shown, includes components similar or identical to the components of system 10 which are identified by the same numbering used in FIGS. I and 7. However, in contrast to system 70, system 80 includes additional electrodes 82 and 84 implanted within duodenum 86 and coupled to IPG 16 via leads 83 and 85, respectively.

In operation, implantable system 80 delivers electrical stimulation to duodenum 86 via electrodes 82, 84 in addition to restricting a portion of stomach 8 and delivering electrical stimulation to the restricted portion of stomach 8 via electrodes 14. As a result, system 80 may more completely address the contributing factors to obesity. In particular, delivering electrical stimulation to duodenum 86 may further increase gastric motility thereby reducing caloric absorption from the food ingested by patient 2 or, alternatively, delay gastric emptying thereby inducing a sensation of nausea or fullness in patient 2 more quickly.

The electrodes 82, 84 carried at the distal end of each of leads 83 and 85 may be attached to duodenum 86 in a variety of ways. For example, electrodes 82 and 84 may be surgically sutured onto duodenum 86 or fixed by penetration or anchoring devices, such as hooks, barbs, or helical structures within the tissue of duodenum 86. Surgical adhesives may also be used to attach electrodes 82 and 84 to duodenum 86. In any case, electrodes 82 and 84 are implanted in acceptable electrical contact with duodenum 86.

In some embodiments, electrical stimulation may be delivered to duodenum 86 of patient 2 via a second gastric constriction device with integrally formed electrodes. In this case, the second gastric constriction device may be implanted and function similar to gastric constriction device 12 with integrally formed electrodes 14 discussed throughout this disclosure. The electrodes of the second gastric constriction device may be coupled to controller 16 and deliver stimulation to duodenum similar to electrodes 82, 84, i.e., in a time-interleaved or sequential manner with electrodes 14. The degree of gastric constriction of the second gastric constriction device may be adjusted to delay gastric emptying. Hence, two or more gastric constriction devices may be used at different positions in the gastrointestinal tract on a coordinated basis to retriction intake or delay emptying and apply electrical stimulation.

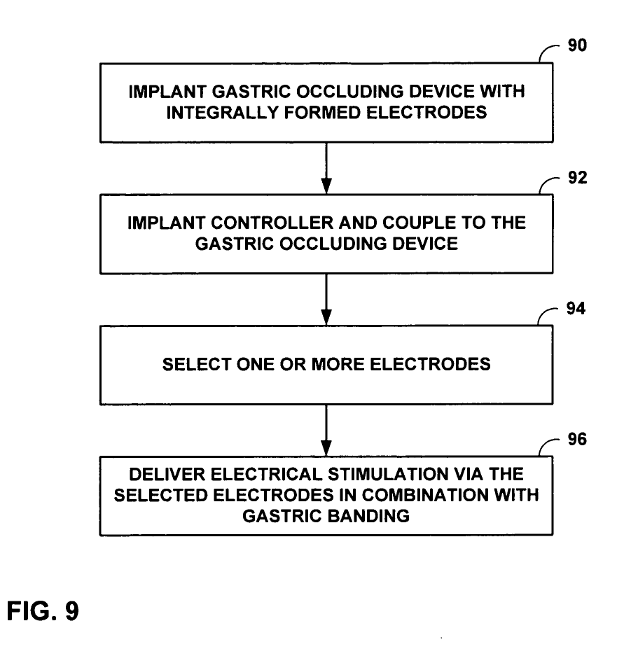

FIG. 9 is a flow chart illustrating a technique for delivering electrical stimulation to a patient in combination with gastric banding using system 10. Initially, gastric constriction device 12, i.e., a mechanical or hydraulic gastric band with a plurality of electrodes integrally formed in the band, is implanted within patient 2 (90). Typically, gastric constriction device 12 can be inserted through a laparoscopic cannula to completely encircle and partition a portion of stomach 8 into an upper stomach 8A and lower stomach 8B thereby restricting the passage of food into lower stomach 8B. As an example, gastric constriction device 12 may be implanted by connecting first and second ends 24, 26 together via connection mechanism 15 to achieve a desired inner diameter 58. In some embodiments, gastric constriction device 12 may be implanted as shown in FIG. 1, although gastric constriction device 12 may be implanted at various locations of the gastrointestinal tract.

Next, various well known open or laparoscopic surgical procedures may be used for implanting controller 16 and coupling controller 16 to gastric constriction device 12 (92). Controller 16 may be implanted within a subcutaneous pocket proximate to gastric constriction device 12. The surgeon may couple controller 16 to gastric constriction device 12 by connecting conduit 18 and lead 17 to respective inputs, ports, or connectors on gastric constriction device 12. In particular, conduit 18 couples controller 16 and gastric constriction device 12 so that controller 16 and device 12 are in fluid communication with each other. Lead 17 electrically couples controller 16 to electrodes 14 integrally formed with gastric constriction device 12. As previously described, controller 16 may be substantially smaller in size than a control unit and pulse generator contained within separate housing. As a result, controller 16 may reduce the trauma experienced by patient 2 as a result of the surgical procedure.

When gastric constriction device 12 and controller 16 have been implanted within patient 2 and coupled to each other, a clinician selects one or more electrodes (96) to deliver electrical stimulation to the restricted portion of stomach 8. In general, selecting one or more electrodes includes selecting one or more of electrodes 14 or, more specifically, one or more possible electrode combinations from electrodes 14 and the stimulation parameters for delivering electrical stimulation via the selected electrode combinations.

As previously described, a clinician may test all or at least a combination of all the possible electrode combinations in order to identify an effective combination of electrodes and their polarities. In some cases, the clinician may test electrode combinations by manually specifying each combination or test based on intuition or some idiosyncratic methodology, and record notes on the efficacy and side effects of each electrode combination after delivery in order to later compare and selected from the tested electrode combinations. Alternatively, system 10 may utilize a search algorithm to select electrode combinations to test. In some embodiments, system 10 may receive input from patient 2, for example, by entering input into external programmer 20 in wireless communication with system 10, to indicate preferred electrode combinations.

When an effective, or optimum, electrode combination has been discovered, system 10 delivers electrical stimulation via the selected electrodes in combination with gastric banding (98). The selected electrodes may deliver electrical stimulation in accordance with various modes, e.g., continuously, in a series of bursts, or a combination of both. The selected electrodes may also deliver electrical stimulation according to different stimulation parameters at different times during the day or may even deliver each pulse in accordance with a different set of parameters. When more than one electrode combination is selected to deliver electrical stimulation, each selected electrode combination may deliver electrical stimulation in accordance with a different set of stimulation parameters. The electrode combinations may also deliver electrical stimulation at the same time or on a time-interleaved basis.

By utilizing gastric constriction device 12 to restrict the food intake of patient 2 and deliver electrical stimulation to the restricted portion of stomach 8 via selected electrodes integrally formed with gastric constriction device 12, system 10 may limit food intake and vary increase gastric motility thereby providing a multiple pronged approach for treating obesity. Additionally, enclosing a control unit that controls the degree of gastric constriction and a pulse generator that controls delivery of electrical simulation within a common housing may reduce the cost of the system and reduce the trauma experienced by the patient during and after implantation.

To induce a sensation of satiety or nausea or modulate gastric motility, in general, a train of pulses may be delivered according to the following stimulation parameters: amplitude approximately equal to 1 to 8 volts, pulse width approximately equal to 0.5 to 10 milliseconds, pulse rate approximately equal to 5 to 40 Hz, and an ON/OFF duty cycle approximately equal to 10 to 75 percent. To induce a sensation of satiety of nausea or modulate gastric motility, in general, a series of continuous pulses may be delivered according to the following stimulation parameters: amplitude approximately equal to 1 to 8 volts, pulse width approximately equal to 1 to 20 milliseconds, pulse rate approximately equal to 0.06 to 20 Hz.

Various embodiments of the invention have been described. These and other embodiments are within the scope of the following claims.

1. An implantable medical device comprising:
a gastric constriction device configured to constrict a portion of a gastrointestinal tract of a patient;
a plurality of electrodes carried by the gastric constriction device;
a housing implanted in the patient; and
a controller within the housing that controls a degree of gastric constriction provided by the gastric constriction device, selects one or more of the electrodes, and delivers electrical stimulation energy to the patient via the selected electrodes.

2. The device of claim 1, wherein the housing is sized for implantation within a subcutaneous pocket of the patient located in one of an abdomen and lower back of the patient.

3. The device of claim 1, wherein the gastric constriction device is configured to encircle a portion of the gastrointestinal tract and partition the portion of the gastrointestinal tract into an upper and a lower region.

4. The device of claim 1, wherein the electrodes are integrally formed with the gastric constriction device such that the electrodes are located circumferentially around the constricted portion of the gastrointestinal tract.

5. The device of claim 1, wherein the electrodes are arranged in a linear array of electrodes that extends along a length of the gastric constriction device.

6. The device of claim 1, wherein the electrodes are arranged in a two-dimensional pattern of electrodes across a surface of the gastric constriction device.

7. The device of claim 1, wherein the controller includes a control unit that adjusts the degree of gastric constriction provided by the gastric constriction device, a pulse generator that generates the electrical stimulation energy, a switch device that selects one or more of the electrodes and couples the stimulation energy to the selected electrodes to deliver the stimulation energy to the patient, and a processor that controls the control unit, the pulse generator, and the switch device.

8. The device of claim 7, wherein the control unit comprises a micro motor that mechanically increases and decreases the degree of gastric constriction provided by the gastric constriction device.

9. The device of claim 7, wherein the control unit comprises a fluid pump that pumps fluid to the gastric constriction device to increase the degree of gastric constriction and pumps fluid from the gastric constriction device to decrease the degree of gastric constriction.

10. The device of claim 7, wherein the processor controls the stimulation generator and the switch device to deliver the stimulation energy to the patient in accordance with a set of stimulation parameters, wherein the stimulation parameters include electrode polarity, stimulation pulse amplitude, stimulation pulse width, and stimulation pulse rate.

11. The device of claim 7, wherein the processor controls the stimulation generator and the switch device to deliver the stimulation energy to multiple selected sets of the electrodes.

12. The device of claim 7, wherein the processor controls the stimulation generator and the switch device to deliver the stimulation energy to multiple selected sets of the electrodes on a time-interleaved basis.

13. The device of claim 7, wherein the switch device selects a first electrode combination that delivers stimulation energy to the patient and a second electrode combination that deliver stimulation energy to the patient on a time-interleaved basis with the stimulation energy delivered via the first electrode combination.

14. The device of claim 7, wherein the first electrode combination delivers stimulation energy to the patient in accordance with a first set of stimulation parameters and the second electrode combination delivers stimulation energy to the patient in accordance with a second set of stimulation parameters.

15. The device of claim 1, further comprising one or more electrodes located separately from the electrodes carried by the gastric constriction device, wherein the stimulation generator delivers stimulation energy to the gastrointestinal tract of the patient via the separately located electrodes.

16. The device of claim 15, wherein the stimulation energy delivered to the electrodes carried by the gastric constriction device is configured to induce a sensation of at least one of nausea or satiety in the patient, and the stimulation energy delivered to the separately located electrode is configured to promote gastric motility.

17. A method comprising:
constricting a portion of a gastrointestinal tract of a patient using a gastric constriction device, wherein the gastric constriction device carries a plurality of electrodes;
delivering electrical stimulation energy to the constricted portion of the gastrointestinal tract via a selected subset of the electrodes; and
controlling the gastric constriction device and the delivery of electrical stimulation energy via a common controller.

18. The method of claim 17, wherein the control is enclosed in a single housing implanted within a subcutaneous pocket of the patient located in one of an abdomen and lower back of the patient.

19. The method of claim 17, wherein the gastric constriction device encircles a portion of the gastrointestinal tract and partitions the portion of the gastrointestinal tract into an upper and a lower region.

20. The method of claim 17, wherein the electrodes are integrally formed with the gastric constriction device such that the electrodes are located circumferentially around the constricted portion of the gastrointestinal tract.

21. The method of claim 17, wherein the electrodes are arranged in a linear array of electrodes that extends along a length of the gastric constriction device.

22. The method of claim 17, wherein the electrodes are arranged in a two-dimensional pattern of electrodes across a surface of the gastric constriction device.

23. The method of claim 17, wherein constricting the portion of the gastrointestinal tract comprises mechanically adjusting the degree of gastric constriction provided by the gastric constriction device via a micro motor.

24. The method of claim 17, wherein constricting the portion of the gastrointestinal tract comprises pumping fluid to the gastric constriction band to increase the degree of gastric constriction and pumping fluid from the gastric constriction device to decrease the degree of gastric constriction.

25. The method of claim 17, further comprising controlling the stimulation energy in accordance with a set of stimulation parameters, wherein the stimulation parameters include electrode polarity, stimulation pulse amplitude, stimulation pulse width, and stimulation pulse rate.

26. The method of claim 17, further comprising delivering the stimulation energy to multiple selected sets of the electrodes on a time-interleaved basis.

27. The method of claim 17, further comprising selecting a first electrode combination that delivers stimulation energy to the patient and a second electrode combination that deliver stimulation energy to the patient on a time-interleaved basis with the stimulation energy delivered via the first electrode combination.

28. The method of claim 27, further comprising delivering the stimulation energy to the first electrode combination in accordance with a first set of stimulation parameters and delivering the stimulation energy to the second electrode combination in accordance with a second set of stimulation parameters.

29. The method of claim 17, further comprising delivering stimulation energy to the patient via one or more electrodes located separately from the electrodes carried by the gastric constriction device.

30. The method of claim 29, wherein the stimulation energy delivered to the electrodes carried by the gastric constriction device is configured to induce a sensation of at least one of nausea or satiety in the patient, and the stimulation energy delivered to the separately located electrode is configured to promote gastric motility.

31. The method of claim 17, wherein the electrodes carried by the gastric constriction device are integrally formed with the gastric constriction device such that at least portions of the electrodes are exposed by an interior surface of the gastric constriction device to couple the stimulation energy to the gastrointestinal tract upon placement of the gastric constriction device within the patient.

32. An external control device for controlling an implantable gastric constriction device and an implantable electrical stimulation generator, the external control device comprising:
a processor that generates control signals to control operation of the implantable gastric constriction device and the implantable electrical stimulation generator; and
a wireless telemetry interface that communicates the control signals to at least one control unit that controls the implantable gastric constriction device and the implantable electrical stimulation generator.

33. A method for controlling an implantable gastric constriction device and an implantable electrical stimulation generator, the method comprising:
generating control signals to control operation of the implantable gastric constriction device and the implantable electrical stimulation generator; and
communicating the control signals by wireless telemetry to at least one control unit that controls the implantable gastric constriction device and the implantable electrical stimulation generator.
